(19) United States
(12) Patent Application Publication
(10) Pub. No.: US 2008/0146994 A1
(43) Pub. Date: Jun. 19, 2008

(54) Retrofittable aspiration prevention mechanism for patients

(76) Inventor: Allen Gerber, High Falls, NY (US)

Correspondence Address: Lawrence D. Cutter, 11 Deer Path Drive, New Paltz, NY, 12561-2813, US

(21) Appl. No.: 12/070,515

(22) Filed: Feb. 19, 2008

Related U.S. Application Data
(63) Continuation-in-part of application No. 11/804,109, filed on May 17, 2007.
Application No. 11/804,109 is a continuation-in-part of application No. 11/545,382, filed on Oct. 10, 2006.

(51) Int. Cl. A61M 5/172

(52) U.S. Cl. 604/66

(57) ABSTRACT

A retrofittable device is employed to remediate the problem of fluid aspiration in patients being fed through a feeding tube. In one embodiment the feeding pump is plugged into the device which is plugged into a power outlet. A patient angle sensor triggers power cutoff to the pump and stoppage of fluid flow. In another embodiment, the device responds to the angle sensor by actuating a flow limiting device which either pinches the tube closed or actuates a controllable valve disposed within the flow path.

### CROSS-REFERENCE TO RELATED APPLICATIONS

The Present application is a continuation-in-part application of patent application Ser. No. 11/804,109 filed on May 17, 2007 which is itself a continuation-in-part of patent application Ser. No. 11/545,382 filed on Oct. 10, 2006. This application contains subject matter which is related to the subject matter of the above-mentioned applications, which is owned by the same entity as the present application.

### TECHNICAL FIELD

The present invention is generally directed to the medical field as it relates to patient care, particularly in a hospital, nursing home or other institutional settings and even in some home care settings. More particularly, the present invention relates in general to systems and methods for preventing aspiration of stomach contents by bed ridden patients connected to feeding tubes and to ancillary functions that may be performed by such devices.

### BACKGROUND OF THE INVENTION

It is well known that millions of people around the world are fed through gastric feeding tubes once they can no longer feed themselves. The most common version of this practice occurs in the use of nasogastric feeding tubes. Other gastric feeding practices include the surgical insertion of a feeding tube directly into the stomach through the abdominal wall (PEG tubes). The present invention is employable in all of these situations in which gastric feeding is provided. Thus, in the appended claims the term gastric tube refers to both nasogastric tubes and to PEG tubes,

While the use of gastric feeding mechanisms is not only a common and a life preserving procedure, complications can arise. In particular, one of these complications is aspiration pneumonia. This condition, which can be life threatening, particularly in older patients or in patients with weakened immune systems, can occur via several mechanisms. A common one of these mechanisms is one in which the patient slides down in bed to an angle which is sufficiently to allow gastric fluids to ascend the esophagus and to be inhaled into the lungs. Typically, this angle is about 30°. When the patient angle in the bed reaches this point, the stomach contents are able to percolate up through the esophagus and down into the lungs. The fact that this is a significant problem in patient care is reflected in the fact that in many states the occurrences of aspirational pneumonia resulting in death are reportable incidents to state oversight authority.

The use of feeding pumps has been around for several decades. However, the problem of aspiration, while it has been around as long as the use of such devices, has not been addressed by the medical instrumentation arts which have failed to address the problems associated with the use of feeding tubes, feeding pumps and the problems of aspirated materials. The present invention provides a simple retrofittable device which is simple, inexpensive and easy to operate, whether or not implemented using microprocessor control.

It is noted that, while the present invention is principally directed to the problems associated with gastric feeding tubes, nonetheless, it is equally applicable to those situations in which substances other than nourishment are being provided through such a tube.

From the above, it is therefore seen that there exists a need in the art to overcome the deficiencies and limitations described herein and above.

### SUMMARY OF THE INVENTION

Accordingly, in order to solve these problems, there is provided a mechanical or electronic device that senses when a patient slides down below a predetermined angle. The device is operable in one of two ways or in both ways. In a first embodiment, the detection of improper patient angle shuts off power being supplied to the feeding device. This embodiment is implemented by interposing a controllable power switch between an electrically powered feeding pump and the A/C wall outlet or other source from which the pump is being powered. In a second embodiment of the present invention, the detection of improper patient angle triggers a motor, relay, or solenoid with a mechanical actuator which squeezes the feeding tube with sufficient pressure to stop the flow of material within the tube. This latter modality of operation is best suited for use with pumps that include a battery backup for purposes of safety during power failures.

In preferred embodiments of the present invention, the device is controlled via a microprocessor. The use of a microprocessor provides the ability to more closely monitor and detect patient activity, control, angle sensor and to provide additional functions, such as determining that a patient has fallen.

The sensing of patient position below a certain angle may also be used to alert the attending medical staff that a patient is in an undesirable position. Additionally, the detection of an undesirable patient angle may also be employed to automatically raise the head and/or foot portion of an adjustable bed so as to prevent further downward sliding.

Additional features and advantages are realized through the techniques of the present invention. Other embodiments and aspects of the invention are described in detail herein and are considered a part of the claimed invention.

Accordingly, it is an object of the present invention to reduce and/or eliminate the problem of aspiration in patients connected to gastric tubes.

It is also an object of the present invention to reduce and/or eliminate the problem of exposing portions of the esophagus to gastric fluids.

It is a still further object of the present invention to provide medical staff with an indication of undesired patient movement.

It is yet another object of the present invention to provide a feedback mechanism for raising the foot portion of a patients bed to prevent further sliding.

Additional features and advantages are realized through the techniques of the present invention. Other embodiments and aspects of the invention are described in detail herein and are considered a part of the claimed invention.

The recitation herein of desirable objects which are met by various embodiments of the present invention is not meant to imply or suggest that any or all of these objects are present as essential features, either individually or collectively, in the most general embodiment of the present invention or in any of its more specific embodiments.

### BRIEF DESCRIPTION OF THE DRAWINGS

The subject matter which is regarded as the invention is particularly pointed out and distinctly claimed in the concluding portion of the specification. The invention, however, both as to organization and method of practice, together with the further objects and advantages thereof, may best be understood by reference to the following description taken in connection with the accompanying drawings in which:

FIG. 1 is a side elevation view indicating the relative positions of a patient and a reclinable bed, and particularly indicating the angle of the bed;

FIG. 2 is a stylized, side elevation view of a patient showing the stomach and esophagus for a patient reclining at the angle shown in FIG. 1, as well as showing the placement of an angle sensor;

FIG. 3 is a block diagram illustrating the system and method of the present invention. FIG. 4 is a block diagram view similar to FIG. 3 but more particularly illustrating the presence of a stomach content quantity sensor;

FIG. 4 is a block diagram view similar to FIG. 3 but more particularly illustrating the presence of a stomach content quantity sensor;

FIG. 5 is an enlarged view of a portion of FIG. 2, which more particularly illustrates an embodiment of the present invention employing a stomach content sensor;

FIG. 6 is a schematic diagram illustrating the use of a girth sensor for providing a fullness signal;

FIG. 7 is a schematic diagram similar to FIG. 5 but more particularly illustrating the use of a PEG tube;

FIG. 8 is a diagram illustrating an exemplary flow control algorithm based on both patient angle and fullness sensor;

FIGS. 9 and 10 are block diagrams illustrating the various components of the present invention, with FIG. 10 illustrating a wireless version; and

FIG. 11 is a process flow diagram illustrating an exemplary algorithm which implements one embodiment of the present invention.

### DETAILED DESCRIPTION

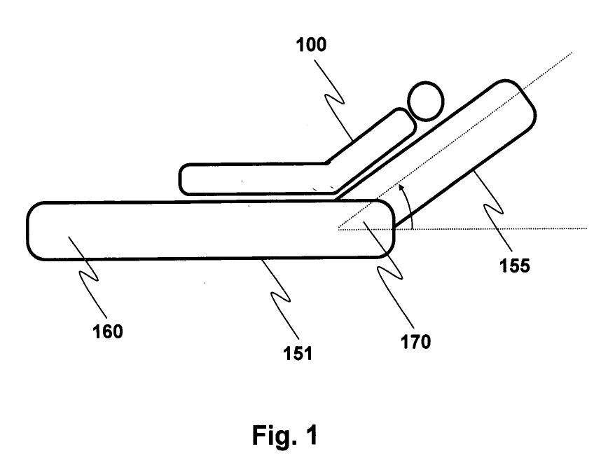

FIG. 1 illustrates the environment in which the present invention is employed. In particular, there is shown patient 100 positioned in a reclining position on bed 150 which includes movable head portion 155 and which may also include a likewise movable foot portion 160 which is employed either for patient comfort or for elevation of the lower extremities. It is also seen the patient is reclining at angle 170 with respect to the horizontal. Reference to the horizontal is employed herein for measurement and determination of improper angle since the “horizontal” is really determined by gravity and it is gravity that is the principal driver of gastric fluid into the esophagus and beyond.

It is noted herein that the angle shown in FIG. 1 is the angle of the adjustable head portion of the bed with respect to the horizontal portion of the bed. Even though the illustration suggests it, FIG. 1 does not reflect the fact that the position of a patient who has slid down in the bed. It should also be noted that the beds of concern herein may also be equipped with adjustable foot portion 160 as well. In fact, if it is detected that patient 100 is sliding down in the bed, the adjustable foot portion of the bed may be raised to prevent further sliding. This is an optional feature of the present invention.

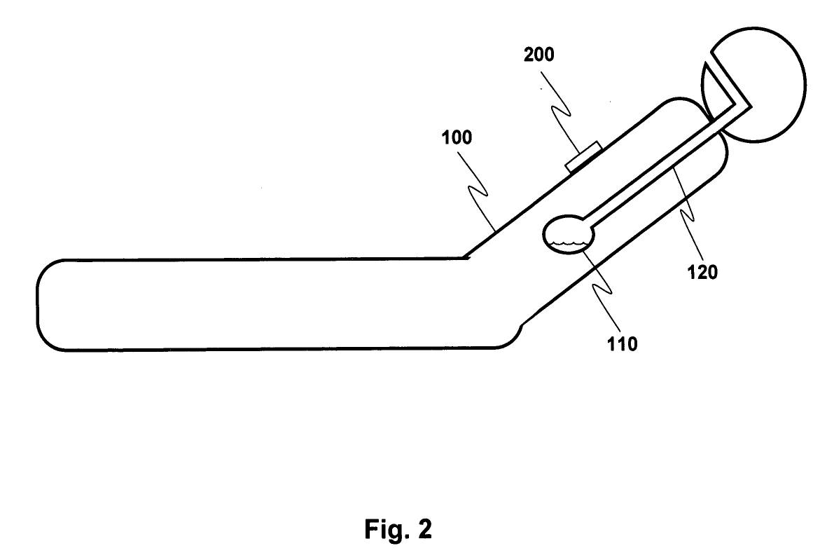

FIG. 2 provides a greater detail of the situation being considered with respect to patient 100 and the specific problem that is solved. Basic human anatomy teaches that stomach 110 is connected to esophagus 120. It is easily seen that if the patient's angle is low, that is, if the patient is closer to a horizontal position, stomach contents can enter esophagus 120 simply by gravity flow. The problems associated with this flow are discussed above, but, needless to say, it is not a desirable situation.

Additionally, FIG. 2 illustrates the placement of sensor 200. Sensor 200 is preferably placed on the chest of patient 200. It is affixed to the patient or to the patient or to the patient's clothing by any convenient means (though the latter is not preferred since clothing position is not always a good indicator of patient angle or position). For short term use adhesive material on one side of sensor 200 holds it in place. For use with clothing or gowns, a wider range of options is available for affixing the sensor, including pins, elastic bands and Velcro™. These latter two items may also be employed to affix the sensor more firmly to the patient. Sensor 200 comprises any convenient mechanism for sensing angle. At its simplest it comprises a mercury filled insulative container with electrical contacts being closed when it contact with the mercury. The interior shape of the container is such that the mercury becomes in contact with the contacts at a predetermined angle. The sensor may also include adjustable exterior flaps to provide a selectable angle. It is noted, however, that there is a wide range of sensors and sensor technology which may be employed. For example, one could employ a ball or other sliding or rolling interior object which either makes electrical contact or which is of sufficient weight to cause switch contacts to close. Additionally, the interior moving object may be employed to interrupt light falling on a photocell. Magnetic or other optical sensors may be employed as well. In fact, any device which implements the generation of an electrical or even electromagnetic signal based on dependence on an angle with respect to feeding tube flow is employable. As indicated, the sensor may even comprise a wireless device which transmits an activation signal to pump control 220. More sophisticated sensors 200 which actually provide a signal indicative of the actual angle, as opposed to the angle merely exceeding a threshold value are also employed in the present invention. With a more sophisticated indication of angle being provided, it is then possible to provide an early warning indication of a patient sliding downward. In such cases, the alarm to patient or staff is variable in intensity depending on the angular degree sensed.

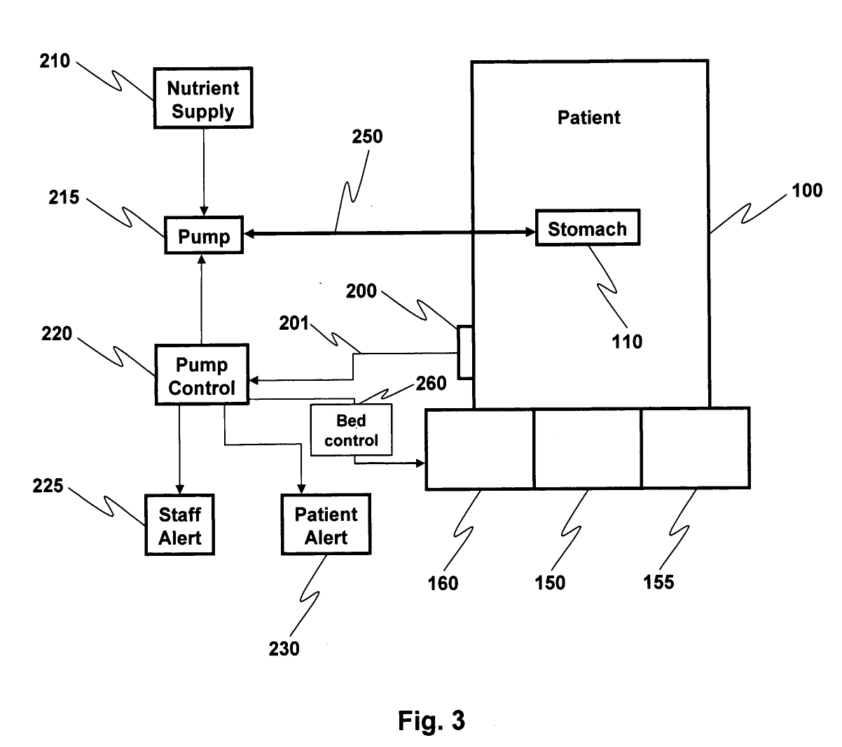

The solution to the aspiration problem is shown in greater detail in FIG. 3. In particular, sensor 200, which is affixed to patient 100, sends a signal to pump control 220 which, in normal operation, sends nutrient materials from supply 210 to stomach 110 of patient 100. If patient 100 slides down in bed 150 to an undesired, predetermined angle, sensor 200 signals pump control 220 to shut off the supply of nutrient or other material to stomach 110. Additionally, the system is provided with an optional feature in which gastric fluid is actually pulled back into gastric tube 250. In this regard, note the two directions indicated for tube 250.

It is also seen that the signal from sensor 200 is also capable of providing an audible or visual signal 225 to hospital staff members to alert them that patient 100 has slid down into bed 150 to an undesirable and possibly unsafe position. Pump control 220 may also be used to supply an audible, visual or vibratory signal 230 to patient 100 as a mechanism for immediate correction by the patient himself or herself, if possible. This same signal from sensor 200 may also be used to control bed 150. In particular, in conjunction with a bed control unit (not shown), sensor 200 is also seen to be capable of providing an actuation signal to cause foot portion 160 of bed 150 to raise so as to forestall further sliding.

In the discussion above, it is assumed that nutrients are provided through a gastric tube via a pump which acts as a positive control element in the system. However, it is noted that it is also possible that nutrient supply 210 may be positioned above the patient so that it is supplied by gravitational action. In this case, the role of “pump” 220 is less “active” in that it operates not so much as a pump but as a valve to control the rate of flow. In such an arrangement the optional feature of pump reversal is not available. However, apart from this drawback, the present invention is equally capable of operating with gravity flow systems.

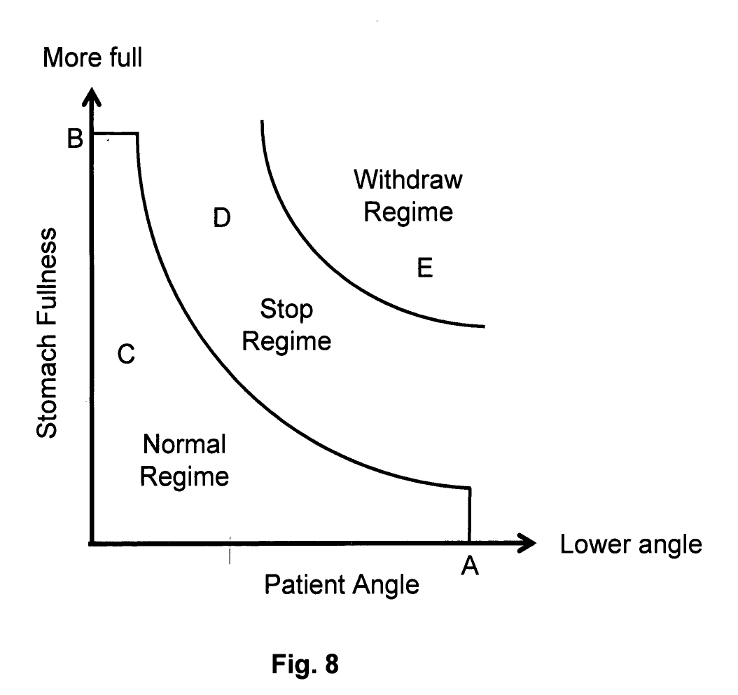

Pump control 220 is provided by any convenient mechanism. Application specific integrated circuit (ASIC) chips may be employed, off-the shelf control components may be used or pump control 220 may be implemented via any standard microprocessor or microcontroller. An exemplary control algorithm based on sensed patient angle and patient stomach content level is shown in FIG. 8.

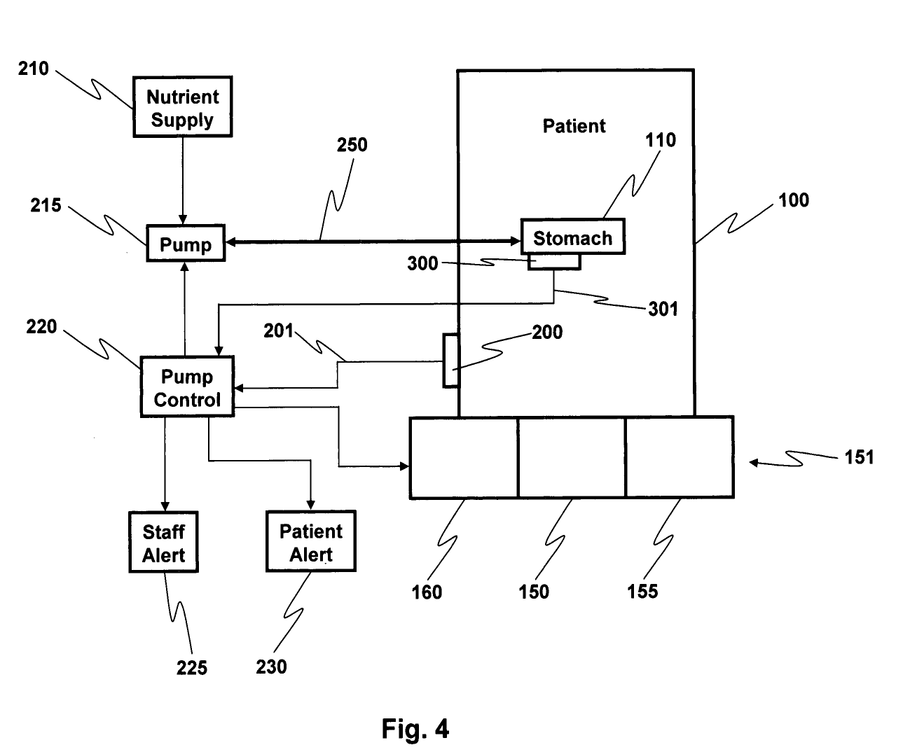

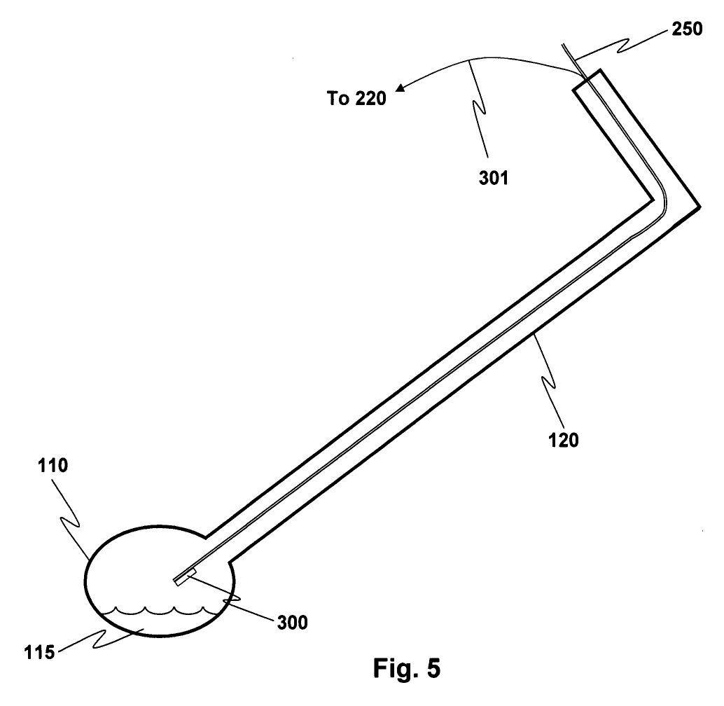

FIG. 4 is similar to FIG. 3 but it more particularly illustrates the presence of an additional mechanism which is capable of providing an indication of the quantity of material within the stomach at any given time. In particulate, one form of fullness sensor 300 is disposed at the end of feeding tube 250 as shown in FIG. 5. When implemented in this fashion, fullness sensor 300 has connected thereto signal wire or cable 301 which is typically disposed alongside feeding tube 250 or may be manufactured along with it as an integral assembly. Wire or cable 301 is provided to pump control 220 to be used, either alone or in conjunction with a signal from angle sensor 200, to control the flow of fluid in feeding tube 250, either stopping it, or in some cases, actually reversing the flow.

Fullness sensor 300, as shown in FIG. 5 may comprise an electrical circuit whose properties change when in contact with gastric fluid 115. Fullness sensor 300 may also respond to being in contact with any liquid; it may respond to being in contact with a liquid of a certain acidity; or fullness sensor 300 may respond to the level of liquid present. Additionally, fullness sensor 300 may also include ultrasonic transmission and receiving components which produce a signal which is proportional to or a function of unoccupied gastric volume. In this way, if a known volume of fluid is introduced into the stomach in a known amount of time, ultrasonic fullness sensor 300 provides “before” and “after” signals which can be used to indicate the change in stomach volume as a percentage which occurs as the result of the input of a known volume in a known amount of time. In this way, stomach volume can be calculated and the sensor can be calibrated accordingly. Fullness sensor 300 may also comprise a pressure transducer which responds to elevated levels of gas pressure within the stomach.

Exterior ultrasound measurements produced using readily available equipment may also be employed as a mechanism for determining fullness and the need to either stop or withdraw fluid. This approach, however, typically has the disadvantage of requiring human intervention and is harder to automate.

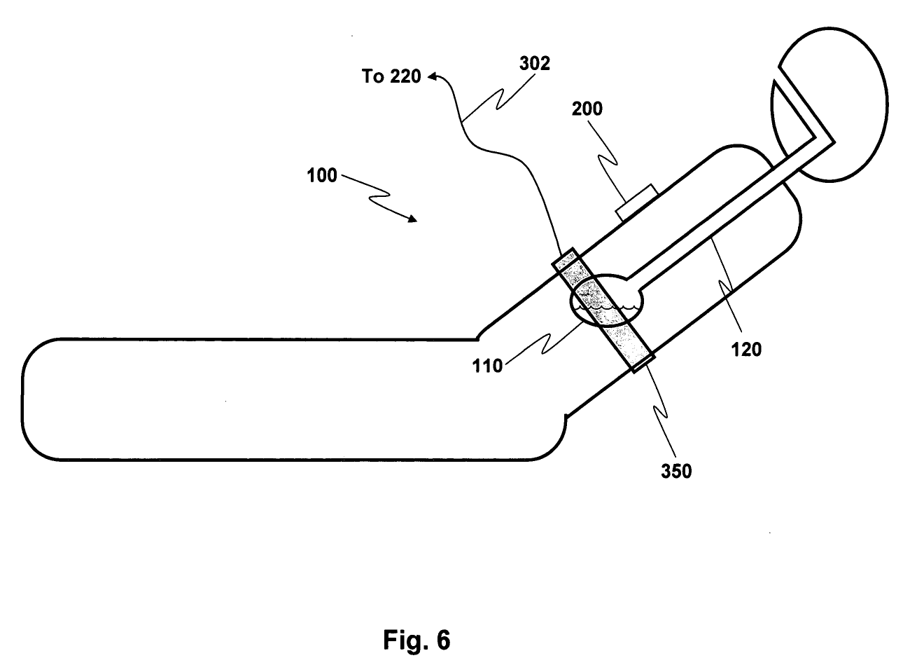

FIG. 6 illustrates the situation in which girth sensor 350 is employed as a mechanism for determining stomach fullness and/or changes in stomach fullness. Girth sensor 350 is disposed about the patient's abdomen as shown and lead 302 is supplied to pump control 220. In the event that girth sensor 350 includes a wireless transmission device, electrical conductor 302 is not necessary.

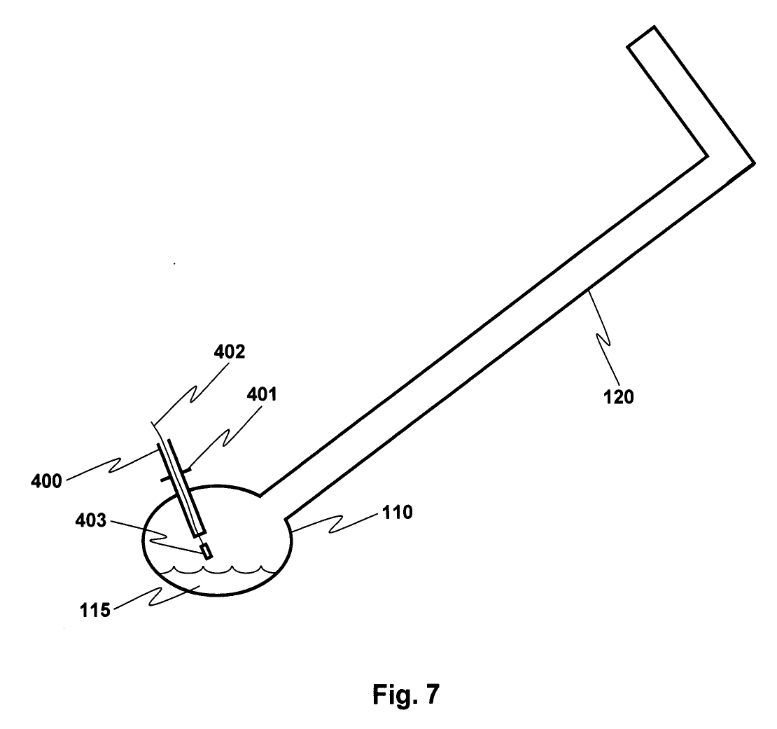

FIG. 7 illustrates the use of the present invention when, instead of a nasogastric tube, PEG tube 400 is employed. Such tubes typically include collar portion 401 which is disposed against the abdomen and is affixed thereto in a sealed fashion to guard against providing a passage for infection. Fullness sensor 403 is disposed through PEG tube 400 and is coupled externally through electrical conductor 402.

FIG. 8 represents an exemplary algorithm for pump control and/or stoppage control (the latter being especially in the case of a gravity driven nutrient supply) based jointly on patient angle and patient stomach fullness. In the case of each variable, it is seen that there is a point reached where some action is taken such as when the patient angle gets too low (point A in FIG. 8) or when the patient's stomach contents become too full (point B), this latter point being particularly desirable in the implementation of a method designed to keep stomach contents out of the esophagus, independent of patient angle. Also shown in FIG. 8 is region C which illustrates normal operation in a region of relatively high patient angle and low stomach contents. As these variables change in a direction away from the illustrated origin, control enters a control regime D in which feeding or nutrition flow is stopped. Further excursions of these variables in a direction away from the indicated origin result in flow control entering region E characterized not just by flow stoppage but by flow reversal. As should be fully appreciated, variations of the regions illustrated in FIG. 8 are not only possible to achieve specific purposes in particular patients but it is also easily possible to implement any diagram such as that shown using microprocessors with the given curves stored in its memory in a number of convenient forms.

The present invention is preferably provided with a dual axis accelerator and/or inclinometer such as the ADIS16003 model as provided by Analog Devices, Inc. In this regard, it is noted that patient angle sensors per se appear to have been described in U.S. Pat. No. 4,348,562 issued to Robert E. Florin and issued on Sep. 7, 1982. However, the use of this angle sensor is limited to the detection of conditions leading to patient falls. Since the present invention includes the use of a controller and a more sophisticated sensor, the present invention preferably also includes an alarm function unrelated to the desire to halt the flow of fluid in a feeding (or other) tube. For example, it is known that patients sometimes aspirate food, phlegm or saliva even if they are not currently being fed with a feeding tube. In those circumstances in which a feeding tube is disconnected, even temporarily, the present invention is still preferably kept in place to monitor patient angle to prevent aspiration of material unrelated to the feeding tube situation. This is particularly true for stroke patients, but for many other types of patients as well.

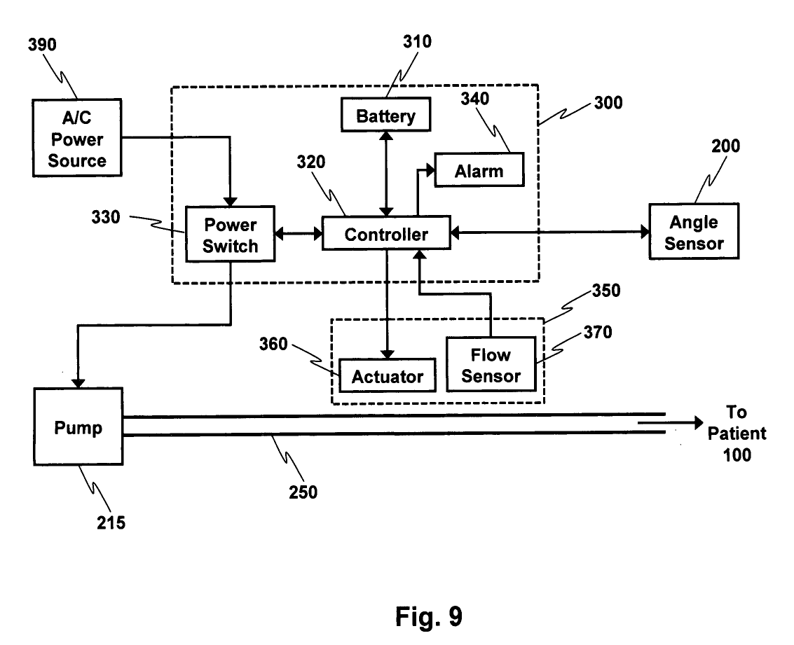

FIG. 9 illustrates an embodiment of the present invention which is retrofittable and/or used in conjunction with currently available feeding pumps and related devices. In particular, it is seen that the embodiment shown in FIG. 9 shows the invention as main package 300 which is for example plugged into source of electrical power 390. Pump 215, which is to be controlled by the present invention, it is then preferably connected to an electrical outlet interface provided on main package 300. Two other connections are made to package 300 in the deployment of the present invention. In particular, angle sensor 200 is disposed attached to an electrical cable which also plugs into package 300. Package 300 further includes any convenient form of pluggable electrical cable for connecting to actuator 360 disposed in separate package 350 which optionally also includes flow sensor 370. Actuator 360 is disposed so as to at least partially surround feeding tube 250 and which is activated by controller 320 to squeeze tube 250 to prevent further flow of nutrients or medication to patient 100. It should also be understood that sensor 200 and controller housing 300 may be provided as a single integrated component. However, in such cases it is desirable that A/C power level components such as power switch 330 be disposed outside of this housing.

Package 300 includes controller 320 which is implemented in the form of a controller such as the well-known and programmable PIC controller (model no. PIC16F877A). In anticipation of operation during power failure conditions controller 320 is powered by batteries 310 also contained within package 300. In response to a signal from angle sensor 200, controller 320 operates to shut off power to pump 215 through control of power switch 330. It is noted however that certain feeding pumps are provided with their own battery backup so that simply disconnecting these units from a source of electrical power does not actually prevent their continuing to function. In such cases, preferred embodiments of the present invention operate by detecting continued flow in the feeding tube 250 using flow sensor 370 which is preferably disposed in the same package 350 as actuator 360. Actuator 360 and flow sensor 370 represent portions of the present invention which are disposed adjacent to feeding tube 250 and which are preferably connected to the package 300 the a single electrical cable (unlike the units shown in FIG. 9 which are not intended to illustrate detailed physical configurations but rather functional configurations).

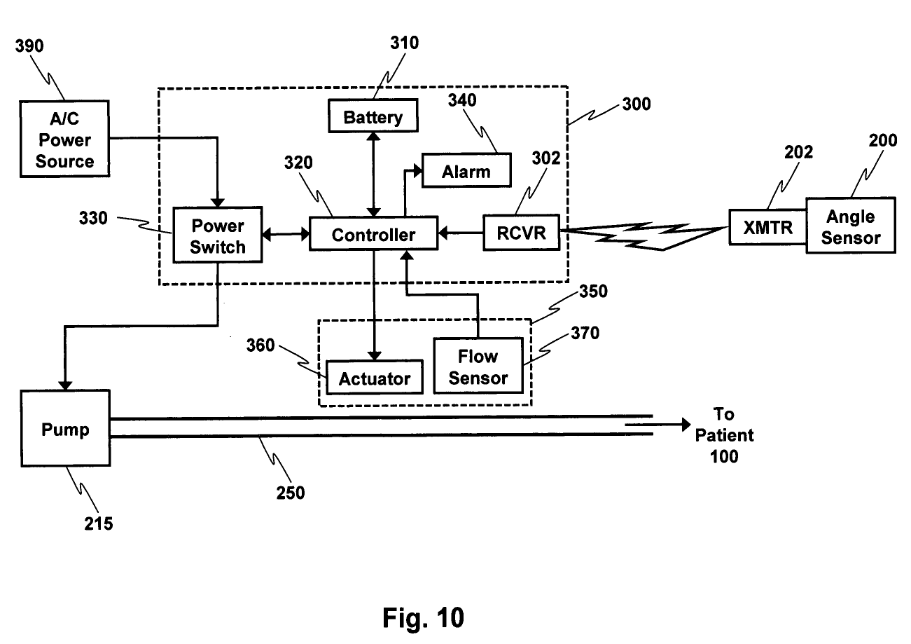

In the event that signals from angle sensor 200 to controller 320 indicate the presence of an improper angle or other patient positioning irregularity, controller 320 respondents by causing actuator 360 to exert sufficient pressure on feeding tube 250 to prevent the continued flow of fluid therein. Flow sensor 370, which is optional, is employed in those circumstances where it is desirable to provide feedback to the controller of the present invention indicating that flow has indeed ceased. In some embodiments of the present invention, for use in those circumstances where it is known that a pump includes a battery backup, power switch 330 is either eliminated or simply bypassed with actuator 360 being relied upon to produce a cessation of fluid flow. This arrangement provides easy retrofit capabilities. An arrangement in which the feeding tube is cut and an electrically actuatable valve is inserted in the flow path also provides a retrofit capability but is not quite as convenient. As shown in FIG. 10, it is also possible to provide sensor 200 with wireless transmitter 202 and to likewise provide controller package 300 with wireless receiver 302. Package unit 300 and package unit 350 are also connectable in a wireless manner. In any event actuator 360 operates to cutoff fluid flow in tube 250 by mechanical intervention separate and apart from any operating modalities of the pump supplying the fluid.

In any event, as indicated elsewhere herein, the present invention also preferably includes an alarm function 340 being controlled by controller 320 in response to signals sent from angle sensor 200. Alarm 340 is either an audio alarm or a visual alarm or both. Additionally, alarm 340 also is capable of including wireless transmission functions capable of broadcasting either processed or raw information from angle sensor 200.

According to the description shown in FIG. 9, the following components provide the core components of the present invention which are easily integrated to provide the functionality described herein. In particular, there is included a dual axis accelerometer/inclinometer which provides a dual-axis acceleration and inclination angle measurement system packaged as an integrated circuit which is deployed to provide varying resolution of the patient's position relative to the Earth's horizon. The sensor is firmly affixed to the patient. Any convenient attachment method may be provided. For example, the sensor may be provided with an adhesive backing (temporary or otherwise) such as the adhesive that is employed with EKG electrodes. Alternatively, the sensor may be provided with an adjustable elastic loop which fits around the patient's chest or upper body. There is also included a microcontroller functioning as a dedicated controller which is deployed to enable the function of sampling input from the accelerometer/inclinometer and for controlling pump and alarm activity. Power relays, switches and communications components, which are driven by the microcontroller, attached relays and switches are used to stop, start, or change the pump's modality and trigger alerts based on monitored parameters and encoded heuristics. It is therefore seen that the present invention is capable of monitoring the duration of a significant event and is capable of responding accordingly. The patient may shift position for a brief period of time exceeding a threshold. The microcontroller is programmed to respond based on rules and logic that take into account variations in the patient's position as a function of time.

Is also seen that the present invention is capable of using as accelerometer/inclinometer's acceleration outputs as parameters that indicate the velocity of the patients change in position. If the acceleration the patient's position is sudden, an alarm is preferably triggered to alert hospital staff that the patient may have fallen; as indicated elsewhere herein this is an ancillary benefit of the present invention that exists in addition to controlling pump functions.

The specific angle sensing and feedback control mechanisms deployed herein are a function of the microcontroller chosen. In any event, the algorithm provided is employed to interpret changes in the patient's position and trigger one or more relays to activate a bed controller if desired or convenient. Any industry standard embedded microprocessor is employable to input and interpret analog or digital information provided by an accelerometer/inclinometer attached to a patient. Command and data transfer between the inclinometer (sensor) and the microprocessor are either wired or wireless. An algorithm is employed to provide varying responses to changes in the patient's position and control various attached components.

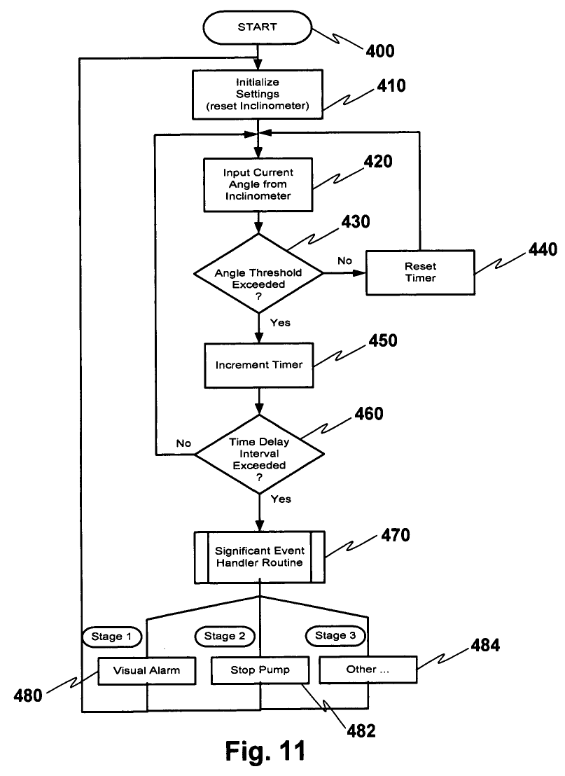

Included in FIG. 11 is a sample process flow diagram that illustrates a possible primary use-case scenario for responding to significant events with respect to a patient's position as a function of time.

The patient's position and incline are continuously monitored by the microcontroller 320. Microcontroller 320 reads the X and Y tilt and acceleration vectors. Microcontroller 320 assesses these values against a predefined heuristic to manage the systems response to various events.

Positioning the sensor on the patient is accomplished by attaching the sensor to the patient's garment, on the shoulder or on the upper body. Positioning of the sensor is not critical. Once attached, microcontroller 320 is initialized to sense the current X and Y coordinates relative to the Earth's horizon as a baseline reference. All changes in the patient's position are interpreted relative to this baseline.

Microcontroller 320 is programmed to respond to multiple disparate events and inputs. Its primary role is to monitor and interpret changes in the patient's position as it continuously reads the position of the accelerometer/inclinometer (sensor). Microcontroller 320 is programmed to automatically control the pump and other discrete devices based on a heuristic algorithm which preferably emulates how an attending technician/care provider would manually responded to a similar significant event. Automatic pump shut-down, pump reversal (in those cases in which the control circuitry is linked with the design of the pump, as opposed to being retrofitted as provided herein), auto-recovery and graduated alerting of staff are all possible functions of microcontroller 320.

FIG. 11 illustrates an algorithm that is implemented by controller 320. The following steps are provided as part of a usable control procedure: Start: System switched on (step 400). Initialize Setting (Reset Inclinometer): Set baseline angle after affixing sensor to patient and initialize timer (step 410). Input Current Angle from Inclinometer: Request current X and Y axis positions from sensor 200 (step 420). Angle Threshold Exceeded?: If the current patient's incline is within tolerances, reset the timer (step 440) and request new input (step 420). Reset Timer: The timer is reset as the result of X and Y axis parameters are within normal range. If either the X or Y axis incline angle exceeds a limit indicating an abnormal condition, increment the timer (step 450). Time Delay Interval Exceeded?: (step 460) If the duration of the event did not exceed a time limit, return to step 420 and request new input from the sensor. If the timer (counter) has exceeded the acceptable time limit, the significant event handler routine (step 470) is invoked. Significant Event Handler Routine: The event handler routine is called if a significant event occurs requiring a corrective action or alert. A set of conditional statements evaluate key parameters, such as the severity of the angle of incline and the duration of the event to determine if one or more staged responses are indicated. Stage 1-Stage 3 (steps 480, 482 and 484): The event handler routine (step 470) selects the appropriate sub-routine for taking corrective action. Once remediation is complete, the system is reset (step 410).

While the invention has been described in detail herein in accordance with certain preferred embodiments thereof, many modifications and changes therein may be effected by those skilled in the art. Accordingly, it is intended by the appended claims to cover all such modifications and changes as fall within the spirit and scope of the invention.

What is claimed is:

1. A device to prevent aspiration of gastric fluids in a patient being fed or medicated through a gastric tube, said device comprising:
an angle sensor affixable to said patient, said sensor being capable of providing an electrical signal indicative of the sensor's being angularly positioned beyond a threshold angle;
an electrical control circuit for receiving said signal and for cutting off power to a feeding pump causing material to flow in said tube.

2. The device of claim 1 in which said feeding pump is selected from the group consisting of pumps which do not include a battery backup and pumps in which a battery backup is disabled.

3. The device of claim 1 which is pluggable into a wall outlet

4. The device of claim 3 in which said device includes a socket into which a power cord for said pump is pluggable.

5. The device of claim 1 in which said angle sensor includes more than one axis.

6. The device of claim 1 in which said sensor includes means for affixation to said patient.

7. The device of claim 1 in which said control circuit actuates an alarm to alert attending staff.

8. The device of claim 1 in which said control circuit actuates an alarm to alert said patient.

9. The device of claim 1 in which said angular threshold is adjustable.

10. The device of claim 1 in which said sensor includes a wireless transmitter to supply said signal to said control circuit which includes a receiver for said signal.

11. A device to prevent aspiration of gastric fluids in a patient being fed or medicated through a gastric tube, said device comprising:
an angle sensor affixable to said patient, said sensor being capable of providing an electrical signal indicative of the sensor's being angularly positioned beyond a threshold angle;
an electrical control circuit for receiving said signal and for actuating a flow limiting device which cuts off flow of fluid within said tube.

12. The device of claim 11 in which said flow limiting device operates to urge a moveable member against said feeding tube to cut off flow of fluid therein.

13. The device of claim 11 in which said flow limiting device is an electrically actuatable valve disposed within the flow path of said tube.

14. The device of claim 11 in which said control circuit and said flow control device are connected wirelessly.
